(19) United States
(12) Patent Application Publication
(10) Pub. No.: US 2005/0113406 A1
(43) Pub. Date: May 26, 2005

(54) Polymorphs of clopidogrel hydrochloride and their use as antithrombic compounds

(76) Inventors: Peter Kotay Nagy, Vac (HU); Jozsef Barkoczy, Budapest (HU); Gyula Simig, Budapest (HU); Zsuzsa Szent Kirallyi, Budapest (HU); Tamas Gregor, Csomor (HU); Bela Farkas, Veszprem (HU); Gyorgyi Vereczkeyne Donath, Budapest (HU); Kalman Nagy, Budapest (HU); Gyulane Kortvelyessy, Budapest (HU)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/504,042

(22) PCT Filed: Dec. 20, 2002
(86) PCT No.: PCT/HU02/00157

(30) Foreign Application Priority Data
Feb. 6, 2002 (HU) P 0200438

(51) Int. Cl. C07D 498/02; A61K 31/4743

(52) U.S. Cl. 514/301; 546/114

(57) ABSTRACT

The invention relates to crystalline forms I and II methyl-(S)-(+)-(2-chlorophenyl)-2-(6,7-dihydro-4H-thieno[3,2.c]pyridine-5-yl)-acetate hydrochloride of the Formula (I) and hydrates thereof, a process for the preparation thereof and pharmaceutical compositions containing the same. The new polymorphs according to the invention exhibit blood platelet aggregation inhibiting and antithrombotic effect.

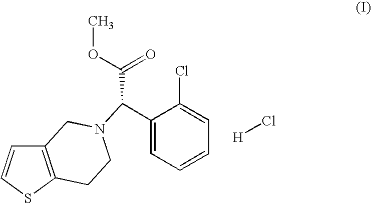

### FIELD OF THE INVENTION

This invention relates to new polymorphs of clopidogrel hydrochloride, a process for the preparation thereof, pharmaceutical compositions comprising said new polymorphs and the use of the new polymorphs for blood platelet aggregation inhibiting and antithrombotic treatment.

More particularly the invention is concerned with new crystalline forms I and II methyl-(S)-(+)-(2-chlorophenyl)-2-(6,7-dihydro-4H-thieno[3,2-c]pyridine-5-yl)-acetate hydrochloride of the Formula 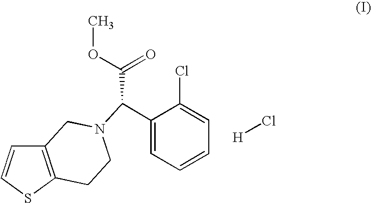
and hydrates thereof, a process for the preparation of the new polymorphs, pharmaceutical compositions comprising said new polymorphs and the use of the new polymorphs for blood platelet aggregation inhibiting and antithrombotic treatment.

### TECHNICAL BACKGROUND

Methyl-(S)-(+)-(2-chlorophenyl)-2-(6,7-dihydro-4H-thieno[3,2-c]pyridine-5-yl)-acetate hydrogen sulfate is a known blood platelet aggregation inhibitory and antithrombotic pharmaceutical active ingredient having the INN (International Non-Proprietory Name) clopidogrel hydrogen sulfate.

Clopidogrel hydrogen sulfate was first described in EP 281,459 corresponding to HU 197,909. Methyl-(S)-(+)-(2-chlorophenyl)-2-(6,7-dihydro-4H-thieno[3,2-c]pyridine-5-yl)-acetate hydrochloride was also first disclosed in this patent specification. According to said patent the hydrochloride salt is prepared by dissolving clopidogrel base in diethyl ether and precipitating the salt with diethyl ether containing hydrogen chloride. In the patent specification methyl-(S)-(+)-(2-chlorophenyl)-2-(6,7-dihydro-4H-thieno[3,2-c]pyridine-5-yl)-acetate hydrochloride is characterized by its melting point of 117° C. and optical rotation of [α]_{D}²⁰=+62.23° (c=1.82, methanol). The patent is, however, completely silent in disclosing the crystal form of the product and IR or X-ray powder diffraction data characterizing the crystalline form are not described either.

According to HU 215,957 methyl-(S)-(+)-(2-chlorophenyl)-2-(6,7-dihydro-4H-thieno[3,2-c]pyridine-5-yl)-acetate hydrochloride is prepared in a similar way by dissolving clopidogrel base in diethyl ether and precipitating the salt with diethyl ether containing hydrogen chloride. In the patent the product is characterized by a protracted melting point interval of 130-140° C. and an optical rotation of [α]_{D}²⁰=+63° (c=1, methanol). There is no teaching of the crystal form of the product and IR or X-ray powder diffraction data characteristic of the product are not disclosed either.

According to WO 98/51681, WO 98/51682, WO 98/51689 and WO 2000/27840 methyl-(S)-(+)-(2-chlorophenyl)-2-(6,7-dihydro-4H-thieno[3,2-c]pyridine-5-yl)-acetate hydrochloride is prepared by dissolving methyl-(S)-(+)-(2-chlorophenyl)-2-(6,7-dihydro-4H-thieno[3,2-c]pyridine-5-yl)-acetate base in diethyl ether, introducing anhydrous gaseous hydrogen chloride into the solution and isolating the crystals formed by filtration. The hydrogen chloride salt is characterized by a melting point of 130-132° C. and an optical rotation of [α]_{D}²⁰=+60°. There is no disclosure of the crystal form of the product, and the IR or X-ray powder diffraction data characteristic of the product are not mentioned either.

Thus clopidogrel hydrochloride of uniform crystal form has not been described in prior art. On the other hand there is a strong need in pharmaceutical industry for active ingredients of uniform morphology. It is known that various polymorphs differ from each other significantly in their important properties (e.g. dissolution speed, bioavailability, chemical stability). Also from a technological point of view there is a strong need for morphologically uniform pharmaceutical active ingredients which can be produced in a reproducible manner on industrial scale too, because the working-up and processing properties of the various polymorphs (e.g. filtrability, drying, solubility, readiness to be compressed into tablets) differ from each other significantly.

### SUMMARY OF THE INVENTION

It is the object of the present invention to provide clopidogrel hydrochloride polymers having uniform morphology meeting the above requirements which can be manufactured in a reproducible manner on industrial scale too.

The above object is solved by the new crystalline methyl-(S)-(+)-(2-chlorophenyl)-2-(6,7-dihydro-4H-thieno[3,2-c]pyridine-5-yl)-acetate hydrochloride polymorphs of the present invention.

The present invention is based on the surprising recognition that two new uniform crystalline forms of clopidogrel hydrochloride can be prepared in a reproducible manner as described below. The melting point of the new polymorphs of the present invention is significantly different from that of the data disclosed in prior art.

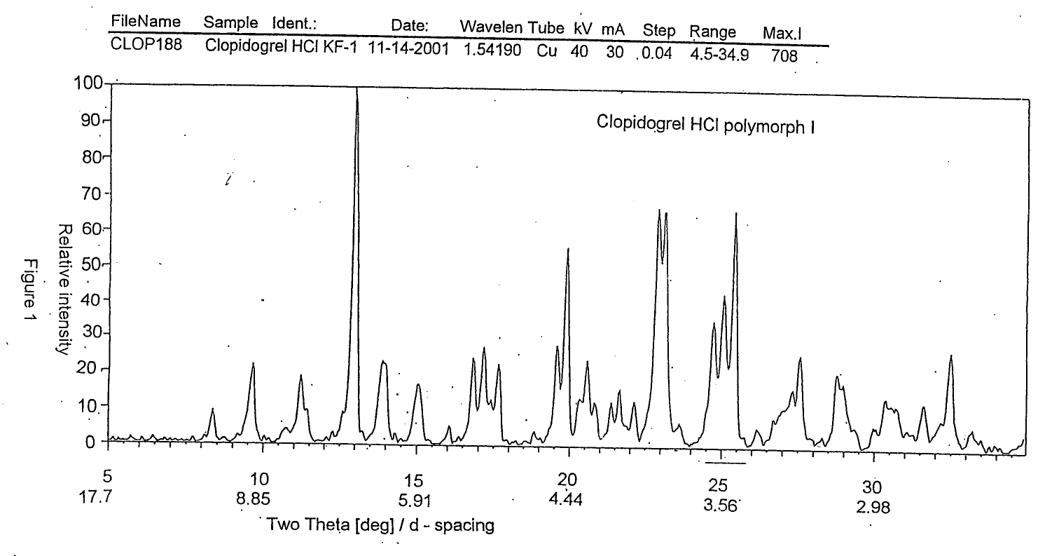

According to the present invention there is provided new crystalline form I methyl-(S)-(+)-(2-chlorophenyl)-2-(6,7-dihydro-4H-thieno[3,2-c]pyridine-5-yl)-acetate hydrochloride of the Formula I and hydrates thereof characterized by the X-ray powder diffraction pattern expressed in Table 1 and FIG. 1.

TABLE 1
Position of diffraction lines and relative intensities.
(>15% of polymorph I)

| Peak | 2*th | D(hkl) | I(abs) | I(rel) |
|---|---|---|---|---|
| No. | [deg] | [Ĺ] | [cts] | [%] |
| 1 | 9.64 | 9.1707 | 152 | 21.51 |
| 2 | 11.22 | 7.8873 | 129 | 18.25 |
| 3 | 12.98 | 6.8222 | 708 | 100 |
| 4 | 13.89 | 6.3759 | 161 | 22.76 |
| 5 | 15.05 | 5.8888 | 115 | 16.25 |
| 6 | 16.82 | 5.2697 | 168 | 23.66 |
| 7 | 17.16 | 5.1661 | 189 | 26.76 |
| 8 | 17.65 | 5.0261 | 156 | 22.06 |
| 9 | 19.58 | 4.5336 | 193 | 27.33 |
| 10 | 19.88 | 4.4654 | 393 | 55.56 |
| 11 | 20.57 | 4.3180 | 165 | 23.37 |
| 12 | 21.64 | 4.1075 | 107 | 15.16 |
| 13 | 22.90 | 3.8841 | 476 | 67.21 |
| 14 | 23.13 | 3.8455 | 469 | 66.30 |
| 15 | 24.73 | 3.6006 | 245 | 34.67 |
| 16 | 25.06 | 3.5540 | 302 | 42.62 |
| 17 | 25.41 | 3.5059 | 471 | 66.49 |
| 18 | 27.31 | 3.2655 | 111 | 15.73 |
| 19 | 27.55 | 3.2372 | 179 | 25.25 |
| 20 | 28.78 | 3.1021 | 143 | 20.16 |
| 21 | 28.97 | 3.0822 | 123 | 17.39 |
| 22 | 32.48 | 2.7570 | 190 | 26.77 |

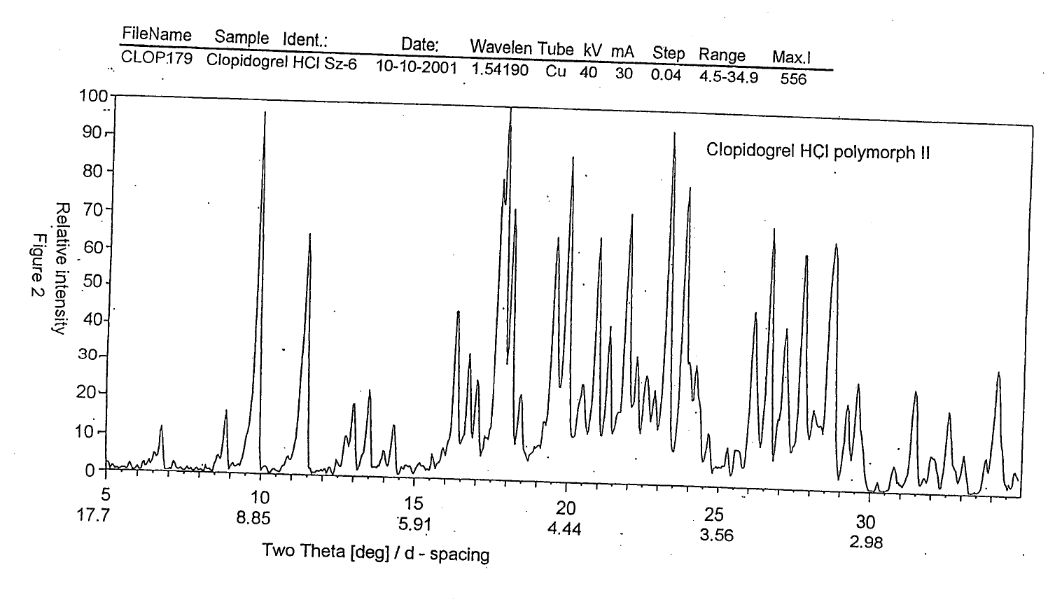

According to the present invention there is also provided new crystalline form II methyl-(S)-(+)-(2-chlorophenyl)-2-(6,7-dihydro-4H-thieno[3,2-c]pyridine-5-yl)-acetate hydrochloride of the Formula I and hydrates thereof characterized by the X-ray powder diffraction pattern expressed in Table 2 and FIG. 2.

TABLE 2
Position of diffraction lines and relative intensities
(>15% of polymorph I)

| Peak | 2*th | D(hkl) | I(abs) | I(rel) |
|---|---|---|---|---|
| No. | [deg] | [Ĺ] | [cts] | [%] |
| 1 | 8.85 | 9.9923 | 88 | 15.83 |
| 2 | 9.85 | 8.9800 | 535 | 96.22 |
| 3 | 11.41 | 7.7575 | 358 | 64.39 |
| 4 | 12.97 | 6.8260 | 101 | 18.17 |
| 5 | 13.49 | 6.5640 | 123 | 22.12 |
| 6 | 16.31 | 5.4362 | 248 | 44.60 |
| 7 | 16.73 | 5.2988 | 182 | 32.73 |
| 8 | 17.01 | 5.2128 | 142 | 25.54 |
| 9 | 17.69 | 5.0139 | 446 | 80.22 |
| 10 | 17.85 | 4.9693 | 556 | 100 |
| 11 | 18.09 | 4.9039 | 403 | 72.48 |
| 12 | 18.44 | 4.8103 | 123 | 22.12 |
| 13 | 19.53 | 4.5455 | 363 | 65.29 |
| 14 | 19.93 | 4.4547 | 482 | 86.69 |
| 15 | 20.46 | 4.3407 | 140 | 25.18 |
| 16 | 20.92 | 4.2457 | 365 | 65.65 |
| 17 | 21.33 | 4.1662 | 232 | 41.73 |
| 18 | 21.92 | 4.0546 | 401 | 72.12 |
| 19 | 22.25 | 3.9956 | 184 | 33.09 |
| 20 | 22.58 | 3.9386 | 156 | 28.06 |
| 21 | 22.85 | 3.8920 | 134 | 24.10 |
| 22 | 23.26 | 3.8250 | 523 | 94.06 |
| 23 | 23.80 | 3.7386 | 443 | 79.68 |
| 24 | 24.21 | 3.6759 | 173 | 31.12 |
| 25 | 26.10 | 3.4143 | 260 | 46.76 |
| 26 | 26.62 | 3.3491 | 388 | 69.78 |
| 27 | 27.14 | 3.2862 | 238 | 42.81 |
| 28 | 27.72 | 3.2189 | 350 | 62.95 |
| 29 | 28.09 | 3.1768 | 113 | 20.32 |
| 30 | 28.67 | 3.1141 | 369 | 66.37 |
| 31 | 29.21 | 3.0573 | 123 | 22.12 |
| 32 | 29.54 | 3.0237 | 155 | 27.88 |
| 33 | 31.42 | 2.8475 | 148 | 26.62 |
| 34 | 32.54 | 2.7515 | 117 | 21.04 |
| 35 | 34.13 | 2.6270 | 180 | 32.37 |

### DETAILED DESCRIPTION OF TH INVENTION

The powder diffraction pattern of new crystaline polymorph I is determined under the following conditions:

Equipment: PHILIPS-XPERT PW 3710 powder

diffractometer
Radiation: CuKα (λ: 1.54190L)
Monochromator: graphite
Exciting voltage: 40 kV
Anode current: 30 Ma
   Method:
Standard reference substance: SRM 675
Mica Powder (synthetic fluorographite), Ser. No.: 981307.
The measurement is continuous: Θ)/2Θ) scan: 4.5°-35.00° 2Θ)
Step size: 0.04°
Sample: surface plain, width 0.5 mm, in quartz sample holder, measured and stored at room temperature.

According to the present invention there is also provided a process for the preparation of crystalline form I methyl-(S)-(+)-(2-chlorophenyl)-2-(6,7-dihydro-4H-thieno[3,2-c]pyridine-5-yl)-acetate hydrochloride of the Formula I and hydrates thereof which comprises
a) dissolving methyl-(S)-(+)-(2-chlorophenyl)-2-(6,7-dihydro-4H-thieno[3,2-c]pyridine-5-yl)-acetate in a dipolar aprotic solvent, or in a less polar aprotic solvent, or in a polar solvent or in a mixture thereof, admixing the solution with a solution of hydrogen chloride formed with a dipolar aprotic solvent, or a less polar aprotic solvent, or a polar solvent or a mixture thereof and isolating the crystaline form I polymorph; or
b) recrystallizing methyl-(S)-(+)-(2-chlorophenyl)-2-(6,7-dihydro-4H-thieno[3,2-c]pyridine-5-yl)-acetate hydrochloride from a dipolar aprotic solvent, or a less polar aprotic solvent or a mixture thereof.

As dipolar aprotic solvent preferably acetone, acetonitrile, ethyl acetate, or dimethyl formamide or a mixture thereof can be used.

As less polar aprotic solvent preferably dioxane, tetrahydrofurane, diisopropyl ether or a mixture thereof can be used.

As polar solvent preferably lower aliphatic alcohols (e.g. ethanol, n-propanol or 2-propanol) can be used.

According to process a) as solvent particularly advantageously acetone or ethyl acetate or a mixture of acetone and ethyl acetate can be used.

According to process b) as solvent particularly advantageously a mixture of acetone and ethyl acetate can be used.

Process a) can be preferably carried out by dissolving methyl-(S)-(+)-(2-chlorophenyl)-2-(6,7-dihydro-4H-thieno[3,2-c]pyridine-5-yl)-acetate base in one of the above solvents and thereafter admixing the solution with a solution of hydrogen chloride formed with one of the above solvents. Salt formation is preferably carried out at room temperature, whereupon the mixture is cooled. The precipitated crystalline form I polymorph is isolated by filtration or centrifuging, washed and dried.

Process b) can be carried out by recrystallizing methyl-(S)-(+)-(2-chlorophenyl)-2-(6,7-dihydro-4H-thieno[3,2-c]pyridine-5-yl)-acetate hydrochloride from a dipolar aprotic solvent or a less polar aprotic solvent or a mixture thereof. As starting material methyl-(S)-(+)-(2-chlorophenyl)-2-(6,7-dihydro-4H-thieno[3,2-c]pyridine-5-yl)-acetate hydrochloride morphologically non-uniform or amorphous or having crystalline form II can be used. The dissolving of methyl-(S)-(+)-(2-chlorophenyl)-2-(6,7-dihydro-4H-thieno[3,2-c]pyridine-5-yl)-acetate hydrochloride can be carried out under heating, preferably at the boiling point of the reaction mixture. The mixture is filtered, the filtrate cooled to a temperature of about room temperature or allowed to cool. The precipitation of crystals can be optionally promoted by inoculating with a small amount of methyl-(S)-(+)-(2-chlorophenyl)-2-(6,7-dihydro-4H-thieno[3,2-c]pyridine-5-yl)-acetate hydrochloride crystals of crystalline form I. One may proceed advantageously by heating the solution of methyl-(S)-(+)-(2-chlorophenyl)-2-(6,7-dihydro-4H-thieno[3,2-c]pyridine-5-yl)-acetate hydrochloride to the boiling point, cooling the solution first to room temperature and thereafter to a temperature between −20° C. and +15° C., isolating the precipitated crystals by filtration or centrifuging, washing and drying.

According to a further aspect of the present invention there is provided a process for the preparation of crystalline form II methyl-(S)-(+)-(2-chlorophenyl)-2-(6,7-dihydro-4H-thieno[3,2-c]pyridine-5-yl)-acetate hydrochloride of the Formula I and hydrates thereof which comprises dissolving methyl-(S)-(+)-(2-chlorophenyl)-2-(6,7-dihydro-4H-thieno[3,2-c]pyridine-5-yl)-acetate in a dipolar aprotic solvent or a mixture thereof, admixing the solution with a solution of hydrogen chloride formed with an aprotic solvent or a mixture thereof and isolating the crystalline form II polymorph.

As dipolar aprotic solvent preferably acetone, acetonitrile, ethyl acetate or dimethyl formamide or a mixture thereof can be used.

One may proceed particularly advantageously by carrying out the process in a mixture of acetone and ethyl acetate.

According to a preferable form of realization of the process methyl-(S)-(+)-(2-chlorophenyl)-2-(6,7-dihydro-4H-thieno[3,2-c]pyridine-5-yl)-acetate is dissolved in a dipolar aprotic solvent or a mixture thereof, whereupon a solution of hydrogen chloride formed with a dipolar aprotic solvent or a mixture thereof is added. One may proceed particularly advantageously by dissolving methyl-(S)-(+)-(2-chlorophenyl)-2-(6,7-dihydro-4H-thieno[3,2-c]pyridine-5-yl)-acetate base in a mixture of acetone and ethyl acetate and adding ethyl acetate containing hydrogen chloride. Salt formation is carried out preferably at room temperature. The precipitated crystalline form II methyl-(S)-(+)-(2-chlorophenyl)-2-(6,7-dihydro-4H-thieno[3,2-c]pyridine-5-yl)-acetate hydrochloride is isolated by filtration or centrifuging, washed and dried.

According to a still further aspect of the present invention there is provided a pharmaceutical composition comprising as active ingredient crystalline form I or II methyl-(S)-(+)-(2-chlorophenyl)-2-(6,7-dihydro-4H-thieno[3,2-c]pyridine-5-yl)-acetate hydrochloride of the Formula I or a hydrate thereof in admixture with inert solid or liquid pharmaceutical carriers and/or auxiliary agents.

The pharmaceutical compositions according to the present invention can be administered preferably orally or parenterally. The oral compositions may be e.g. tablets, capsules, dragees, solutions, elixirs, suspensions or emulsions. The parenteral pharmaceutical compositions can be preferably intravenous or intramuscular injections.

The pharmaceutical compositions can contain conventional pharmaceutical carriers and/or auxiliary agents. For this purpose e.g. magnesium carbonate, magnesium stearate, talc, lactose, pectine, dextine, starch, gelatine, tragacanth, methyl cellulose, sodium carboxy methyl cellulose, lower melting wax, cocoa butter etc. can be used. Soft gelatine capsule can be often prepared without carrier—depending on the properties of the active ingredient—because the wall of the capsule can function as carrier. The oral compositions may be generally tablets, powders, capsules, pilules, cachets and losenges.

The suppositories contain as carrier e.g. lower melting waxes (e.g. mixtures of fatty acid glycerides or cocoa butter). Suppositories can be prepared by melting the wax and homogenously distributing the active ingredient in the melt wax. The thus obtained melted mixture is poured into moulds of suitable form and size and allowed to solidify under cooling.

The tablets can be prepared by admixing the active ingredient with suitable carriers and pressing the mixture into tablets of suitable form and size.

The powders can be prepared by admixing the finely powdered active ingredient with the finely powdered carrier.

The liquid compositions can be solutions, suspensions or emulsions from which the active ingredient can also be released in a sustained manner. The aqueous or aqueous-propylene glycol solutions are advantageous. The liquid pharmaceutical compositions suitable for parenteral administration can be preferably prepared in the form of an aqueous polyethylene glycol solution.

The aqueous solutions suitable for oral administration can be prepared by dissolving the active ingredient in water, optionally with the addition of suitable stabilizers, thickening agents, colourants and sweeteners.

Aqueous suspensions suitable for oral administration can be prepared by suspending the active ingredient in the presence of a viscous substance (e.g. natural or artificial gums, resins, methyl cellulose, sodium carboxy methyl cellulose or other suspending agents) in water.

Another type of solid pharmaceutical compositions is converted into a liquid formulation immediately before use and is administered orally as a liquid. The liquid compositions can be solutions, suspensions or emulsions which can optionally contain stabilizers, buffers, colourants, natural or artificial sweeteners, dispersing agents, thickening agents etc.

The pharmaceutical compositions according to the present invention can be preferably prepared in the form of dosage units which contain the desired amount of the active ingredient. Dosage units can be put on the market in packaged form which contain suitable separated amounts of the active ingredient (e.g. tablets or capsules in packages or vials, or powders in ampoules). The term “dosage unit” encompasses capsules, tablets, losenges and also the packaging which contains the suitable number of dosage units.

According to a further aspect of the present invention there is provided a process for the preparation of pharmaceutical compositions which comprises admixing crystalline form I or II methyl-(S)-(+)-(2-chlorophenyl)-2-(6,7-dihydro-4H-thieno[3,2-c]pyridine-5-yl)-acetate hydrochloride or a hydrate thereof with pharmaceutically acceptable solid or liquid carriers and/or auxiliary agents and bringing the mixture to a galenic form.

The pharmaceutical compositions according to the present invention are prepared by methods of pharmaceutical industry known per se.

The pharmaceutical compositions according to the present invention may contain in addition to crystalline form I or II methyl-(S)-(+)-(2-chlorophenyl)-2-(6,7-dihydro-4H-thieno[3,2-c]pyridine-5-yl)-acetate hydrochloride or a hydrate thereof further compatible pharmaceutical active ingredients.

The daily dose of crystalline form I or II methyl-(S)-(+)-(2-chlorophenyl)-2-(6,7-dihydro-4H-thieno[3,2-c]pyridine-5-yl)-acetate hydrochloride depends on the circumstances of the given case (e.g. the condition and body weight of the patient, the severeness of the condition to be treated, the mode of administration etc.) and is determined by the physician.

According to a further aspect of the present invention there is provided the use of cystalline form I or II methyl-(S)-(+)-(2-chlorophenyl)-2-(6,7-dihydro-4H-thieno[3,2-c]pyridine-5-yl)-acetate hydrochloride or a hydrate thereof as pharmaceutical active ingredient.

According to a still further aspect of the present invention there is provided the use of crystalline form I or II methyl-(S)-(+)-(2-chlorophenyl)-2-(6,7-dihydro-4H-thieno[3,2-c]pyridine-5-yl)-acetate hydrochloride or a hydrate thereof as pharmaceutical active ingredient having blood platelet aggregation inhibiting and antithrombotic effect.

According to a still further aspect of the invention there is provided a blood platelet aggregation inhibiting and antithrombotic method of treatment which comprises administering to the patient in need of such treatment a therapeutically effective amount of crystalline form I or II methyl-(S)-(+)-(2-chlorophenyl)-2-(6,7-dihydro-4H-thieno[3,2-c]pyridine-5-yl)-acetate hydrochloride or a hydrate thereof.

The advantage of the present invention is that the new methyl-(S)-(+)-(2-chlorophenyl)-2-(6,7-dihydro-4H-thieno[3,2-c]pyridine-5-yl)-acetate hydrochloride polymorphs are of uniform morphology and therefore possess reproducible properties in relation to dissolution velocity, bioavailability, chemical stability, working-up and processing (filtrability, drying, tabletting properties etc.). The new polymorphs of the present invention can be prepared by a process which is readily reproducible on industrial scale too.

Further details of the present invention are to be found in the following Examples without limiting the scope of protection to said Examples.

### EXAMPLE 1

### Preparation of Crystalline Form I methyl-(S)-(+)-(2-chlorophenyl)-2-(6,7-dihydro-4H-thieno[3,2-c]pyridine-5-yl)-acetate Hydrochloride

3.21 g of methyl-(S)-(+)-(2-chlorophenyl)-2-(6,7-dihydro-4H-thieno[3,2-c]pyridine-5-yl)-acetate are dissolved in 35 ml of tetrahydrofurane whereupon a solution of tetrahydrofurane saturated with gaseous hydrogen chloride is added. The reaction mixture is stirred at room temperature for 2 hours and thereafter allowed to stand in a refrigerator for 16 hours. The precipitated snow-white crystals are filtered off and washed with cold tetrahydrofurane. Thus 2.6 g of the title compound are obtained. Yield 75%. Mp.: 140-141° C.

### EXAMPLE 2

### Preparation of Crystalline Form I methyl-(S)-(+)-(2-chlorophenyl)-2-(6,7-dihydro-4H-thieno[3,2-c]pyridine-5-yl)-acetate Hydrochloride

3.21 g of methyl-(S)-(+)-(2-chlorophenyl)-2-(6,7-dihydro-4H-thieno[3,2-c]pyridine-5-yl)-acetate are dissolved in a mixture of 4 ml of acetone and 5 ml of ethyl acetate, whereupon 1.3 g of 2-propanol containing hydrogen chloride (31 g HCl/100 g solution) are added. The reaction mixture is stirred at room temperature for 2 hours and thereafter allowed to stand in a refrigerator for 16 hours. The precipitated snow-white crystals are filtered off and washed with cold ethyl acetate. Thus 2.92 g of the title a compound are obtained. Yield 85%. Mp.: 140-141° C.

### EXAMPLE 3

### Preparation of Crystalline Form I methyl-(S)-(+)-(2-chlorophenyl)-2-(6,7-dihydro-4H-thieno[3,2-c]pyridine-5-yl)-acetate Hydrochloride

3.21 g of methyl-(S)-(+)-(2-chlorophenyl)-2-(6,7-dihydro-4H-thieno[3,2-c]pyridine-5-yl)-acetate are dissolved in 2.0 ml of ethyl acetate, whereupon 2.5 g of ethyl acetate containing hydrogen chloride are added (14 g HCl/100 g solution). The reaction mixture is stirred at room temperature for 2 hours and thereafter allowed to stand in a refrigerator for 16-hours. The precipitated snow-white crystals are filtered off and washed with cold tetrahydrofurane. Thus 2.92 g of the title compound are obtained. Yield 85%. Mp.: 140-141° C.

### EXAMPLE 4

### Preparation of Crystalline Form I methyl-(S)-(+)-(2-chlorophenyl)-2-(6,7-dihydro-4H-thieno[3,2-c]pyridine-5-yl)-acetate Hydrochloride

3 g of methyl-(S)-(+)-(2-chlorophenyl)-2-(6,7-dihydro-4H-thieno [3,2-c]pyridine-5-yl)-acetate hydrochloride are dissolved in 40 ml of boiling tetrahydrofurane. The hot solution is filtered, cooled slowly to room temperature under stirring, then stirred at room temperature for 2 hours and allowed to stand in a refrigerator for 16 hours. The precipitated snow-white crystals are filtered off and washed with cold tetrahydrofurane. Thus 2.5 g of the title compound are obtained. Yield 70%. Mp.: 140-141° C.

### EXAMPLE 5

### Preparation of Crystalline Form I methyl-(S)-(+)-(2-chlorophenyl)-2-(6,7-dihydro-4H-thieno[3,2-c]pyridine-5-yl)-acetate Hydrochloride

3 g of methyl-(S)-(+)-(2-chlorophenyl)-2-(6,7-dihydro-4H-thieno[3,2-c]pyridine-5-yl)-acetate hydrochloride are dissolved in a boiling mixture of 70 ml of acetone and 30 ml of diisopropyl ether. The hot solution is filtered, cooled slowly to room temperature under stirring, then stirred at room temperature for 2 hours and allowed to stand in a refrigerator for 16 hours. The precipitated snow-white crystals are filtered off and washed with cold ethyl acetate. Thus 2.2 g of the title compound are obtained. Yield 65%. Mp.: 140-141° C.

### EXAMPLE 6

### Preparation of Crystalline Form I methyl-(S)-(+)-(2-chlorophenyl)-2-(6,7-dihydro-4H-thieno[3,2-c]pyridine-5-yl)-acetate Hydrochloride

3.21 g of methyl-(S)-(+)-(2-chlorophenyl)-2-(6,7-dihydro-4H-thieno[3,2-c]pyridine-5-yl)-acetate are dissolved at room temperature in a mixture of 10 ml acetone and 10 ml of ethyl acetate, whereupon 2.5 g of ethyl acetate containing hydrogen chloride (14 g HCl/100 g solution) are added. The reaction mixture is stirred at room temperature for 16 hours. The precipitated snow-white crystals are filtered off and washed with cold ethyl acetate. Thus 2.2 g of the title compound are obtained. Yield 64°. Mp.: 143-144° C.

1. Crystalline form I methyl-(S)-(+)-(2-chlorophenyl)-2-7-dihydro-4H-thieno[3,2-c]pyridine-5-yl)-acetate hydrochloride of the Formula 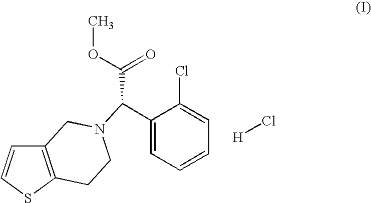
and hydrates thereof characterized by the X-ray powder diffraction pattern expressed in Table 1 and FIG. 1.
TABLE 1
Position of diffraction lines and relative intensities
(>15% of polymorph I)

| Peak | 2*th | D(hkl) | I(abs) | I(rel) |
|---|---|---|---|---|
| No. | [deg] | [Ĺ] | [cts] | [%] |
| 1 | 9.64 | 9.1707 | 152 | 21.51 |
| 2 | 11.22 | 7.8873 | 129 | 18.25 |
| 3 | 12.98 | 6.8222 | 708 | 100 |
| 4 | 13.89 | 6.3759 | 161 | 22.76 |
| 5 | 15.05 | 5.8888 | 115 | 16.25 |
| 6 | 16.82 | 5.2697 | 168 | 23.66 |
| 7 | 17.16 | 5.1661 | 189 | 26.76 |
| 8 | 17.65 | 5.0261 | 156 | 22.06 |
| 9 | 19.58 | 4.5336 | 193 | 27.33 |
| 10 | 19.88 | 4.4654 | 393 | 55.56 |
| 11 | 20.57 | 4.3180 | 165 | 23.37 |
| 12 | 21.64 | 4.1075 | 107 | 15.16 |
| 13 | 22.90 | 3.8841 | 476 | 67.21 |
| 14 | 23.13 | 3.8455 | 469 | 66.30 |
| 15 | 24.73 | 3.6006 | 245 | 34.67 |
| 16 | 25.06 | 3.5540 | 302 | 42.62 |
| 17 | 25.41 | 3.5059 | 471 | 66.49 |
| 18 | 27.31 | 3.2655 | 111 | 15.73 |
| 19 | 27.55 | 3.2372 | 179 | 25.25 |
| 20 | 28.78 | 3.1021 | 143 | 20.16 |
| 21 | 28.97 | 3.0822 | 123 | 17.39 |
| 22 | 32.48 | 2.7570 | 190 | 26.77 |

2. Process for the preparation of crystalline form I methyl-(S)-(+)-(2-chlorophenyl)-2-(6,7-dihydro-4H-thieno[3,2-c]pyridine-5-yl)-acetate hydrochloride of the Formula I and hydrates thereof which comprises
a) dissolving methyl-(S)-(+)-(2-chlorophenyl)-2-(6,7-dihydro-4H-thieno[3,2-c]pyridine-5-yl)-acetate in a dipolar aprotic solvent, or in a less polar aprotic solvent, or in a polar solvent or in a mixture thereof, admixing the solution with a solution of hydrogen chloride formed with a dipolar aprotic solvent, or a less polar aprotic solvent, or a polar solvent or a mixture thereof and isolating the crystaline form I polymorph; or
b) recrystallizing methyl-(S)-(+)-(2-chlorophenyl)-2-(6,7-dihydro-4H-thieno [3,2-c]pyridine-5-yl)-acetate hydrochloride from a dipolar aprotic solvent, or a less polar aprotic solvent or a mixture thereof.

3. Process according to method a) or b) of claim 2 which comprises using acetone, acetonitrile, ethyl acetate or dimethyl formamide or a mixture thereof as dipolar aprotic solvent.

4. Process according to method a) or b) of claim 2 which comprises using dioxane, tetrahydrofurane, or diisopropyl ether or a mixture thereof as less polar aprotic solvent.

5. Process according to method a) of claim 2 which comprises using a lower aliphatic alcohol, preferably ethanol, n-propanol or 2-propanol as polar solvent.

6. Process according to method a) of claim 2 which comprises using as solvent acetone and/or ethyl acetate.

7. Process according to method b) of claim 2 which comprises using a mixture of acetone and ethyl acetate as solvent.

8. Process according to method a) of claim 2 or claim 6 which comprises carrying out salt formation at room temperature and thereafter cooling the reaction mixture.

9. Process according to method b) of claim 2 which comprises carrying out recrystallization by heating the solution of methyl-(S)-(+)-(2-chlorophenyl)-2-(6,7-dihydro-4H-thieno[3,2-c]pyridine-5-yl)-acetate hydrochloride and thereafter cooling the solution.

10. Process according to claim 9 which comprises heating the solution of methyl-(S)-(+)-(2-chlorophenyl)-2-(6,7-dihydro-4H-thieno[3,2-c]pyridine-5-yl)-acetate hydrochloride to the boiling point of the solvent then cooling to room temperature and thereafter cooling to a temperature between −20° C. and +15° C.

11. Pharmaceutical composition comprising as active ingredient crystalline form I methyl-(S)-(+)-(2-chlorophenyl)-2-(6,7-dihydro-4H-thieno[3,2-c]pyridine-5-yl)-acetate hydrochloride of the Formula I or a hydrate thereof in admixture with inert solid or liquid pharmaceutical carriers and/or auxiliary agents.

12. Process for the preparation of pharmaceutical compositions according to claim 11 which comprises admixing crystalline form I methyl-(S)-(+)-(2-chlorophenyl)-2-(6,7-dihydro-4H-thieno[3,2-c]pyridine-5-yl)-acetate hydrochloride or a hydrate thereof with pharmaceutically acceptable solid or liquid carriers and/or auxiliary agents and bringing the mixture to a galenic form.

13. Crystalline form I methyl-(S)-(+)-(2-chlorophenyl)-2-(6,7-dihydro-4H-thieno[3,2-c]pyridine-5-yl)-acetate hydrochloride or a hydrate thereof for use as pharmaceutical active ingredient.

14. Use of crystalline form I methyl-(S)-(+)-(2-chlorophenyl)-2-(6,7-dihydro-4H-thieno[3,2-c]pyridine-5-yl)-acetate hydrochloride or a hydrate thereof as pharmaceutical active ingredient having blood platelet aggregation inhibiting and antithrombotic effect.

15. Blood platelet aggregation inhibiting and antithrombotic method of treatment which comprises administering to the patient in need of such treatment a therapeutically effective amount of crystaline form I methyl-(S)-(+)-(2-chlorophenyl)-2-(6,7-dihydro-4H-thieno[3,2-c]pyridine-5-yl)-acetate hydrochloride or a hydrate thereof.

16. Crystalline form II methyl-(S)-(+)-(2-chlorophenyl)-2-(6,7-dihydro-4H-thieno[3,2-c]pyridine-5-yl)-acetate hydrochloride of the Formula I and hydrates thereof characterized by the X-ray powder diffraction pattern expressed in Table 2 and FIG. 2.
TABLE 2
Position of diffraction lines and relative intensities
(>15% of polymorph I)

| Peak | 2*th | D(hkl) | I(abs) | I(rel) |
|---|---|---|---|---|
| No. | [deg] | [Ĺ] | [cts] | [%] |
| 1 | 8.85 | 9.9923 | 88 | 15.83 |
| 2 | 9.85 | 8.9800 | 535 | 96.22 |
| 3 | 11.41 | 7.7575 | 358 | 64.39 |
| 4 | 12.97 | 6.8260 | 101 | 18.17 |
| 5 | 13.49 | 6.5640 | 123 | 22.12 |
| 6 | 16.31 | 5.4362 | 248 | 44.60 |
| 7 | 16.73 | 5.2988 | 182 | 32.73 |
| 8 | 17.01 | 5.2128 | 142 | 25.54 |
| 9 | 17.69 | 5.0139 | 446 | 80.22 |
| 10 | 17.85 | 4.9693 | 556 | 100 |
| 11 | 18.09 | 4.9039 | 403 | 72.48 |
| 12 | 18.44 | 4.8103 | 123 | 22.12 |
| 13 | 19.53 | 4.5455 | 363 | 65.29 |
| 14 | 19.93 | 4.4547 | 482 | 86.69 |
| 15 | 20.46 | 4.3407 | 140 | 25.18 |
| 16 | 20.92 | 4.2457 | 365 | 65.65 |
| 17 | 21.33 | 4.1662 | 232 | 41.73 |
| 18 | 21.92 | 4.0546 | 401 | 72.12 |
| 19 | 22.25 | 3.9956 | 184 | 33.09 |
| 20 | 22.58 | 3.9386 | 156 | 28.06 |
| 21 | 22.85 | 3.8920 | 134 | 24.10 |
| 22 | 23.26 | 3.8250 | 523 | 94.06 |
| 23 | 23.80 | 3.7386 | 443 | 79.68 |
| 24 | 24.21 | 3.6759 | 173 | 31.12 |
| 25 | 26.10 | 3.4143 | 260 | 46.76 |
| 26 | 26.62 | 3.3491 | 388 | 69.78 |
| 27 | 27.14 | 3.2862 | 238 | 42.81 |
| 28 | 27.72 | 3.2189 | 350 | 62.95 |
| 29 | 28.09 | 3.1768 | 113 | 20.32 |
| 30 | 28.67 | 3.1141 | 369 | 66.37 |
| 31 | 29.21 | 3.0573 | 123 | 22.12 |
| 32 | 29.54 | 3.0237 | 155 | 27.88 |
| 33 | 31.42 | 2.8475 | 148 | 26.62 |
| 34 | 32.54 | 2.7515 | 117 | 21.04 |
| 35 | 34.13 | 2.6270 | 180 | 32.37 |

17. Process for the preparation of crystalline form II methyl-(S)-(+)-(2-chlorophenyl)-2-(6,7-dihydro-4H-thieno[3,2-c]pyridine-5-yl)-acetate hydrochloride of the Formula I and hydrates thereof which comprises dissolving methyl-(S)-(+)-(2-chlorophenyl)-2-(6,7-dihydro-4H-thieno[3,2-c]pyridine-5-yl)-acetate in a dipolar aprotic solvent or a mixture thereof, admixing the solution with a solution of hydrogen chloride formed with an aprotic solvent or a mixture thereof and isolating the crystalline form II polymorph.

18. Process according to claim 17 which comprises using acetone, acetonitrile, ethyl acetate or dimethyl formamide or a mixture thereof as aprotic solvent.

19. Process according to claim 18 which comprises using a mixture of acetone and ethyl acetate as solvent.

20. Process according to any of claims 17-19 which comprises carrying out salt formation at room temperature.

21. Pharmaceutical composition comprising as active ingredient crystalline form II methyl-(S)-(+)-(2-chlorophenyl)-2-(6,7-dihydro-4H-thieno[3,2-c]pyridine-5-yl)-acetate hydrochloride of the Formula I or a hydrate thereof in admixture with inert solid or liquid pharmaceutical carriers and/or auxiliary agents.

22. Process for the preparation of pharmaceutical compositions according to claim 21 which comprises admixing crystalline form II methyl-(S)-(+)-(2-chlorophenyl)-2-(6,7-dihydro-4H-thieno[3,2-c]pyridine-5-yl)-acetate hydrochloride or a hydrate thereof with pharmaceutically acceptable solid or liquid carriers and/or auxiliary agents and bringing the mixture to a galenic form.

23. Crystalline form II methyl-(S)(+)-(2-chlorophenyl)-2-(6,7-dihydro-4H-thieno[3,2-c]pyridine-5-yl)-acetate hydrochloride or a hydrate thereof for use as pharmaceutical active ingredient.

24. Use of crystalline form II methyl-(S)-(+)-(2-chlorophenyl)-2-(6,7-dihydro-4H-thieno[3,2-c]pyridine-5-yl)-acetate hydrochloride or a hydrate thereof as pharmaceutical active ingredient having blood platelet aggregation inhibiting and antithrombotic effect.

25. Blood platelet aggregation inhibiting and antithrombotic method of treatment which comprises administering to the patient in need of such treatment a therapeutically effective amount of crystalline form II methyl-(S)-(+)-(2-chlorophenyl)-2-(6,7-dihydro-4H-thieno[3,2-c]pyridine-5-yl)-acetate hydrochloride or a hydrate thereof.
